(19) United States
(12) Patent Application Publication
(10) Pub. No.: US 2006/0211687 A1
(43) Pub. Date: Sep. 21, 2006

(54) Potassium salt of an hiv integrase inhibitor

(76) Inventors: Michael Palucki, Hillsborough, NJ (US); David Askin, Warren, NJ (US); Vincent J. Angelico, Edison, NJ (US); Robert M Wenslow Jr, East Windsor, NJ (US)

Correspondence Address: MERCK AND CO., INC, P O BOX 2000, RAHWAY, NJ, 07065-0907, US

(21) Appl. No.: 10/548,781

(22) PCT Filed: Mar. 8, 2004
(86) PCT No.: PCT/US04/06968

§ 371 Date: Sep. 12, 2005

Related U.S. Application Data
(60) Provisional application No. 60/453,896, filed on Mar. 12, 2003.

(51) Int. Cl. A61K 31/54; C07D 471/02

(52) U.S. Cl. 514/222.2; 544/3

(57) ABSTRACT

A potassium salt of Compound A is disclosed, wherein Compound A is of formula (1): Compound A is an integrase inhibitor useful for preventing or treating HIV infection, for delaying the onset of AIDS, and for treating AIDS.

### FIELD OF THE INVENTION

The present invention is directed to a pharmaceutically acceptable potassium salt of an HIV integrase inhibitor, Compound A as defined below. The present invention is also directed processes for preparing a potassium salt of Compound A, pharmaceutical compositions containing the salt, and methods for using the salt.

### BACKGROUND OF THE INVENTION

The HIV retrovirus is the causative agent for AIDS. The HIV-1 retrovirus primarily uses the CD4 receptor (a 58 kDa transmembrane protein) to gain entry into cells, through high-affinity interactions between the viral envelope glycoprotein (gp 120) and a specific region of the CD4 molecule found in T-lymphocytes and CD4 (+) T-helper cells (Lasky L. A. et al., Cell 1987, 50: 975-985). HIV infection is characterized by an asymptomatic period immediately following infection that is devoid of clinical manifestations in the patient. Progressive HIV-induced destruction of the immune system then leads to increased susceptibility to opportunistic infections, which eventually produces a syndrome called ARC (AIDS-related complex) characterized by symptoms such as persistent generalized lymphadenopathy, fever, and weight loss, followed itself by full blown AIDS.

After entry of the retrovirus into a cell, viral RNA is converted into DNA, which is then integrated into the host cell DNA. Integration of viral DNA is an essential step in the viral life cycle. Integration is believed to be mediated by integrase, a 32 kDa enzyme, in three steps: assembly of a stable nucleoprotein complex with viral DNA sequences; cleavage of two nucleotides from the 3′ termini of the linear proviral DNA; and covalent joining of the recessed 3′ OH termini of the proviral DNA at a staggered cut made at the host target site. The fourth step in the process, repair synthesis of the resultant gap, may be accomplished by cellular enzymes.

The compound 5-(1,1-dioxido-1,2-thiazinan-2-yl)-N-{4-fluoro-2-[(methylamino)carbonyl]benzyl}-8-hydroxy-1,6-naphthyridine-7-carboxamide (hereinafter designated herein as “Compound A”) is a potent HIV integrase inhibitor. The structure of Compound A is as follows:

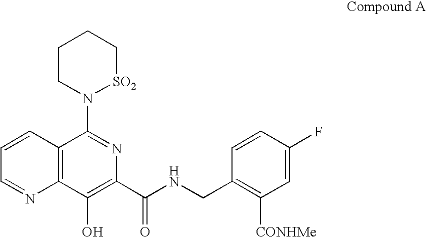

### SUMMARY OF THE INVENTION

The present invention is directed to a potassium salt of Compound A, and particularly to a crystalline potassium salt of Compound A. The potassium salt of Compound A is significantly more soluble in water compared to the free base, and has exhibited improved pharmacokinetics in animal models over the free base. In addition, the potassium salt of Compound A is significantly less hygroscopic and more stable than the sodium salt of Compound A.

The present invention also includes processes for preparing the potassium salt of Compound A and methods of using the Compound A salt for inhibiting HIV integrase, for preventing or treating HIV infection, and for treating or delaying the onset of AIDS.

The foregoing embodiments and other embodiments, aspects and features of the present invention are either further described in or will be apparent from the ensuing description, examples, and appended claims.

### BRIEF DESCRIPTION OF THE DRAWINGS

FIG. 1 is the X-ray powder diffraction pattern for the potassium salt of Compound A as prepared in Example 5.

FIG. 2 is the X-ray powder diffraction pattern for the potassium salt of Compound A as prepared in Example 6.

### DETAILED DESCRIPTION OF THE INVENTION

The present invention provides a pharmaceutically acceptable potassium salt of Compound A, pharmaceutical compositions containing the salt, and methods of making and using the salt. The Compound A potassium salt and pharmaceutical compositions of the present invention are useful for inhibiting HIV integrase, preventing infection by HIV, treating infection by HIV, delaying the onset of AIDS, and treating AIDS, in adults, children or infants. Delaying the onset of AIDS, treating AIDS, or preventing or treating infection by HIV is defined as including, but not limited to, treating a wide range of states of HIV infection: AIDS, ARC, both symptomatic and asymptomatic, and actual or potential exposure to HIV. For example, the potassium salt and pharmaceutical compositions thereof of this invention are useful in treating infection by HIV after suspected past exposure to HIV by, e.g., blood transfusion, exchange of body fluids, bites, accidental needle stick, or exposure to patient blood during surgery. The salts of the invention can also be used in “salvage” therapy; i.e., the potassium salt of Compound A can be used to treat HIV infection, AIDS, or ARC in HIV-positive subjects whose viral load achieved undetectable levels via conventional therapies (e.g., therapies employing known protease inhibitors in combination with one or more known reverse transcriptase inhibitors), and then rebounded due to the emergence of HIV mutants resistant to the known inhibitors.

Compound A is an inhibitor of HIV integrase. Compound A has been tested in an integrase inhibition assay in which strand transfer is catalyzed by recombinant integrase, and has been found to be a potent inhibitor. The strand transfer assay is described in Example 193 of WO 02/30930. Compound A has also been found to be active in an assay for the inhibition of acute HIV infection of T-lymphoid cells conducted in accordance with Vacca et al., Proc. Natl. Acad. Sci. USA 1994, 91: 4096-4100.

The crystalline potassium salt of Compound A has exhibited superior oral bioavailability and improved pharmacokinetics (e.g., improved Cₘₐₓ and AUC) in rats and dogs relative to amorphous and crystalline Compound A. The crystalline potassium salt of Compound A has also exhibited improved stability and less hygrosocpicity than the corresponding sodium salt.

An embodiment of the present invention is a crystalline potassium salt containing a C₁₋₄ alkyl alcohol as a solvate, such as a crystalline potassium salt isopropanolate or a crystalline potassium salt ethanolate. Another embodiment of the present invention is a crystalline potassium salt ethanolate of Compound A. The crystalline ethanolate salt can optionally contain water as a co-solvate, and accordingly still another embodiment is the crystalline potassium salt ethanolate hydrate of Compound A. In an aspect of this embodiment, the crystalline potassium salt ethanolate hydrate is characterized by containing ethanol in an amount in a range of from about 0.3 to about 7.5 wt. % and water in an amount in a range of from about 0.2 to about 5.5 wt. % The amount of ethanol and water co-solvate in the potassium salt of Compound A is typically determined via thermogravimetric analysis.

Another embodiment of the present invention is a crystalline monopotassium salt ethanolate of Compound A, characterized by crystallographic d-spacings of 11.88, 7.45, and 5.07 angstroms. Another embodiment of the present invention is a crystalline monopotassium salt ethanolate of Compound A characterized by crystallographic d-spacings of 11.88, 7.45, 5.07, 4.68, 3.29 and 2.96 angstroms. In an aspect of each of the two preceding embodiments, the K crystalline salt ethanolate of Compound A is a K crystalline-salt ethanolate hydrate (i.e., the crystalline salt contains water as a co-solvate). In a feature of each of these aspects, the K crystalline salt ethanolate hydrate of Compound A is further characterized by containing ethanol in an amount in a range of from about 0.3 to about 7.5 wt. % and water in an amount in a range of from about 0.2 to about 5.5 wt. % In another feature of each of the preceding aspects, the K crystalline salt ethanolate hydrate of Compound A is further characterized by a differential scanning calorimetry (DSC) curve, at a heating rate of 10° C./min in an open cup under nitrogen, exhibiting a first endotherm with a peak temperature of :about 69° C. and an associated heat of fusion of about 4 J/gm, a second endotherm with a peak temperature of about 166° C. and an associated heat of fusion of about 86 J/gm, and a third endotherm with a peak temperature of about 203° C. and an associated heat of fusion of about 4.5 J/gm. While not wishing to be bound by any particular theory, it is believed that the first endotherm is associated with the loss of water, the second endotherm with loss of labile ethanol, and the third endotherm with the loss of more tightly bound ethanol.

Still another embodiment of the present invention is an anhydrous, non-solvated crystalline monopotassium salt of Compound A, characterized by crystallographic d-spacings of 10.40, 10.34 and 5.45 angstroms. Yet another embodiment of the present invention is an anhydrous, non-solvated crystalline monopotassium salt of Compound A, characterized by crystallographic d-spacings of 10.40, 10.34, 5.45, 5.26, 3.96 and 3.52 angstroms. Another embodiment of the present invention is an anhydrous, non-solvated crystalline monopotassium salt of Compound A, characterized by crystallographic d-spacings of 10.40, 10.34, 5.45, 5.26, 3.96, 3.52, 2.72 and 2.58 angstroms. In an aspect of each of the three preceding embodiments, the anhydrous crystalline K salt is further characterized by a differential scanning calorimetry curve, at a heating rate of 10° C./min in a closed cup under nitrogen, exhibiting a sharp endotherm with an onset temperature of about 270° C., a peak temperature of about 272° C., and an associated heat of fusion of about 117 J/gm. Without wishing to be bound by any particular theory, the sharp endotherm is believed to be associated with the melting of the crystal.

The anhydrous, non-solvated crystalline monopotassium salt just described is believed to be a particularly advantageous form of the K salt of the present invention, because it is expected to be stable under a wide range of temperature and humidity conditions and thus easy to formulate with.

The crystallographic d-spacings set forth in the foregoing embodiments can be determined from the XRPD pattern of the crystalline Compound A monopotassium salt.

The present invention includes pharmaceutical compositions comprising a potassium salt of Compound A as originally defined above or as set forth in any of the foregoing embodiments or aspects and a pharmaceutically acceptable carrier.

The present invention also includes pharmaceutical compositions which comprise the product made by combining a potassium salt of Compound A as originally defined above or as set forth in any of the foregoing embodiments or aspects and a pharmaceutically acceptable carrier.

Other embodiments of the present invention include the following:

(a) A method of preventing or treating HIV infection in a subject in need thereof, which comprises administering to the subject a therapeutically effective amount of a potassium salt of Compound A.

(b) A method of delaying the onset of AIDS in a subject in need thereof, which comprises administering to the subject a therapeutically effective amount of a potassium salt of Compound A.

(c) A method of treating AIDS in a subject in need thereof, which comprises administering to the subject a therapeutically effective amount of a potassium salt of Compound A.

(d) A method of inhibiting HIV integrase in a subject in need thereof, which comprises administering to the subject a therapeutically effective amount of a potassium salt of Compound A.

(e) A method of preventing or treating HIV infection in a subject in need thereof, which comprises administering to the subject a pharmaceutical composition comprising a therapeutically effective amount of a potassium salt of Compound A and a pharmaceutically acceptable carrier.

(f) A method of delaying the onset of AIDS in a subject in need thereof, which comprises administering to the subject a pharmaceutical composition comprising a therapeutically effective amount of a potassium salt of Compound A and a pharmaceutically acceptable carrier.

(g) A method of treating AIDS in a subject in need thereof, which comprises administering to the subject a pharmaceutical composition comprising a therapeutically effective amount of a potassium salt of Compound A and a pharmaceutically acceptable carrier.

(h) A method of inhibiting HIV integrase in a subject in need thereof, which comprises administering to the subject a pharmaceutical composition comprising a therapeutically effective amount of a potassium salt of Compound A and a pharmaceutically acceptable carrier,

(i) The method of (a) or (b) or (c) or (d), wherein the potassium salt of Compound A is administered in combination with a therapeutically effective amount of at least one AIDS treatment agent selected from the group consisting of AIDS antiviral agents, immunomodulators, and anti-infective agents.

(j) The method of (a) or (b) or (c) or (d), wherein the potassium salt of Compound A is administered in combination with a therapeutically effective amount of at least one antiviral agent selected from the group consisting of HIV protease inhibitors, non-nucleoside HIV reverse transcriptase inhibitors and nucleoside HIV reverse transcriptase inhibitors.

Additional embodiments of the invention include the methods set forth in (a)-(j) above, wherein the potassium salt of Compound A employed therein is a Compound A potassium salt as set forth in any one of the embodiments or aspects described above.

The present invention also includes a process for preparing a potassium salt of Compound A, which comprises dissolving Compound A in an alcohol or an alcohol-water mixture and treating the resulting solution with a potassium base to form the potassium salt. A suitable solvent for dissolution of Compound A is an alcohol such as a C₁₋₄ alkyl alcohol. In one embodiment, the solvent is isopropanol. In another embodiment, the solvent is ethanol. The solution of Compound A can be formed by adding Compound A to the solvent and then heating the mixture to effect dissolution. Suitable potassium bases include KOH, potassium alkoxides (e.g, potassium C₁₋₄ alkoxides such as the methoxide or ethoxide), potassium amides (e.g., KNH₂), potassium carbonates (e.g., KHCO₃), potassium phosphates, and KH. In one embodiment, the potassium base is KOH. Treatment with KOH typically involves addition of an aqueous solution of KOH to the solution containing Compound A, although KOH in ethanol or KOH in isopropanol can also be employed.

The potassium base (e.g., KOH) can be added to the Compound A solution in any proportion with respect to Compound A which results in the formation of at least some of the desired potassium salt. However, the base is typically added in a proportion which, under the treatment conditions employed (e.g., temperature, degree of agitation), will permit conversion of at least a major portion (and more often substantially all to all) of Compound A to the desired salt. Accordingly, the base is typically added in an amount of from about 0.9 to about 5 equivalents per equivalent of Compound A, and is more typically added in an amount of from about 1 to about 2 equivalents per equivalent of Compound A. In one embodiment, Compound A is dissolved in an alcohol (e.g., ethanol) and treated with from about 1.0 to about 1.3 equivalents of potassium base (e.g., KOH) per equivalent of Compound A.

The treatment of the Compound A solution with the potassium base can be conducted at any temperature at which Compound A is soluble in the chosen solvent. Typically, the treatment step is conducted at a temperature in the range of from about 0 to about 80° C., and more typically at a temperature in the range of from about 20 to about 80° C.

Following the addition of the potassium base (e.g., (KOH), the solution can be aged for a period of time to permit intimate mixing of the base and Compound A. As used herein, the term “aging” and variants thereof (e.g., “aged”) mean allowing the reactants (e.g., KOH and Compound A) to stay in contact for a time and under conditions effective for completion of the reaction. The Compound A solution is optionally agitated (e.g., stirred) during addition of the base and optionally also during any subsequent aging. At the completion of the treatment step, the desired potassium salt can be recovered by filtration, optionally after cooling or concentrating (e.g., by evaporative removal of solvent by the application of heat and/or vacuum) the treated solution.

Embodiments of the processes for preparing a potassium salt of Compound A include any of the preparative processes described above, wherein the potassium salt is crystalline. In each of these embodiments, the Compound A solution can optionally also be seeded with a crystalline K salt of Compound A before, during or subsequent to the addition of the potassium base to promote crystal formation.

Another embodiment of the present invention is a process for preparing a crystalline potassium salt ethanolate of Compound A, which comprises:

(A) dissolving Compound A in ethanol or an ethanol-water mixture to form a solution; and

(B) treating the solution formed in Step A with an aqueous solution of a potassium base (e.g., aqueous KOH) to form the crystalline potassium salt ethanolate of Compound A.

In an aspect of this embodiment, the crystalline potassium salt ethanolate of Compound A formed in Step B is characterized by crystallographic d-spacings of 11.88, 7.45 and 5.07 angstroms. In another aspect, the crystalline potassium salt ethanolate of Compound A formed in Step B is characterized by crystallographic d-spacings of 1 1.88, 7.45, 5.07, 4.68, 3.29 and 2.96 angstroms. In still another aspect of this embodiment, the crystalline salt formed in Step B is a potassium salt ethanolate hydrate of Compound A. In a feature of this aspect, the ethanolate hydrate salt is characterized by the d-spacings 11.88, 7.45 and 5.07 angstroms. In another feature of this aspect, the ethanolate hydrate salt is characterized by the d-spacings 1 1.88, 7.45, 5.07, 4.68, 3.29 and 2.96 angstroms. In sub-features of each of the preceding features, the ethanolate hydrate salt can be further characterized by the DSC endotherms set forth above and/or can be characterized by containing ethanol in an amount in a range of from about 0.3 to about 7.5 wt. % and water in an amount in a range of from about 0.2 to about 5.5 wt. % While not wishing to be bound by any particular theory, it is believed that water and ethanol are co-solvated in the crystalline lattice of Compound A during crystallization, wherein the amount of water present in the lattice is a function of water content of the ethanol during crystallization.

Experiments have been conducted in which the Compound A K salt ethanolate of the present invention was slurried in ethanol containing 1, 2, 3, 4, 5 and 10 vol. % water, followed by measurement of the XRPD patterns of the collected crystals. In addition, the solubility of the Compound A K salt ethanolate was also determined for the 1 to 5 vol. % and 10 vol% water in ethanol solutions. The results showed that when the water content of the ethanol solution exceeded 3%, the XRPD pattern of the crystalline material began to change, and a significant change in the solubility trend and XRPD pattern was observed above 5% water content (i.e., a trend of increasing solubility from 1 to 5 vol % reversed to lower solubility at 10 vol. %). Solid state (SS) NMR studies on the solid materials collected from the slurries have indicated that the collected solids are single-phase materials, not mixed phases (e.g., not a mixture of an ethanolate and a hydrate). Accordingly, it is believed that the crystalline ethanolate hydrate material of the present invention exists as a water-ethanol isomorphic co-solvate. The SS-NMR data have also indicated that the ethanol exists in the crystal lattice in two different solid state environments. In accordance with these experimental results, the crystalline potassium salt ethanolate of Compound A of the invention can contain water as a co-solvate up to a maximum water content, beyond which the crystalline salt (as shown by the XRPD and solubility results described above) changes to another form. Thermogravimetric studies have indicated that the ethanolate hydrate salt of the present invention can contain no more than about 5.5 wt. % solvate water.

The present invention also includes a process for preparing an anhydrous, non-solvated crystalline potassium salt of Compound A, which comprises:

(A) preparing a saturated aqueous solution of a potassium salt of Compound A;

(B) allowing the saturated solution to stand for a time and under conditions effective for crystallization of the anhydrous, non-solvated crystalline salt; and

(C) isolating the crystallized anhydrous salt.

In this process, the saturated solution is typically allowed to stand undisturbed at ambient temperature (e.g., from about 20 to about 30° C.) until crystals begin to form (e.g., in about 3 to 6 months). Additional anhydrous crystalline salt can then be prepared by adding a portion of the original isolated material as seed to a saturated aqueous solution of a K salt of Compound A. Embodiments of this process include the processes in which the isolated crystallized anhydrous salt is characterized by each of the three sets of d-spacings and is optionally further characterized by the DSC endotherm for the anhydrous, non-solvated salt, all as set forth above.

As noted above, the present invention includes pharmaceutical compositions useful for inhibiting HIV integrase, comprising an effective amount of a potassium salt of Compound A and a pharmaceutically acceptable carrier. Pharmaceutical compositions useful for preventing or treating infection by HIV, for delaying the onset of AIDS, or for treating AIDS, are also encompassed by the present invention, as well as a method of inhibiting HIV integrase, and a method of preventing or treating infection by HIV, or delaying the onset of AIDS, or treating AIDS. An aspect of the present invention is a pharmaceutical composition comprising a therapeutically effective amount of a potassium salt of Compound A in combination with a therapeutically effective amount of an agent useful for treating HIV infection and/or AIDS (alternatively referred to as an HIV/AIDS treatment agent) selected from:

(1) an HIV/AIDS antiviral agent,

(2) an anti-infective agent, and

(3) an immunomodulator.

The present invention also includes the use of a potassium salt of Compound A as described above as a medicament for (a) inhibiting HIV integrase, (b) preventing or treating infection by HIV, (c) delaying the onset of AIDS, or (d) treating AIDS. The present invention further includes the use of a potassium salt of Compound A as described above in the preparation of a medicament for (a) inhibiting HIV integrase, (b) preventing or treating infection by HIV, (c) delaying the onset of AIDS, or (d) treating AIDS.

The present invention also includes the use of a potassium salt of Compound A of the present invention as described above in combination with one or more HIV/AIDS treatment agents selected from an HIV/AIDS antiviral agent, an anti-infective agent, and an immunomodulator for use as a medicament for (a) inhibiting HIV integrase, (b) preventing or treating infection by HIV, (c) delaying the onset of AIDS, or (d) treating AIDS, said medicament comprising an effective amount of the potassium salt of Compound A and an effective amount of the one or more treatment agents.

The present invention further includes the use of a potassium salt of Compound A of the present invention as described above in combination with one or more HIV/AIDS treatment agents selected from an HIV/AIDS antiviral agent, an anti-infective agent, and an immunomodulator for the manufacture of a medicament for (a) inhibiting HIV integrase, (b) preventing or treating infection by HIV, (c) delaying the onset of AIDS, or (d) treating AIDS, said medicament comprising an effective amount of the potassium salt of Compound A and an effective amount of the one or more treatment agents.

For the uses described above, a potassium salt of Compound A of the present invention may be administered orally, parenterally (including subcutaneous injections, intravenous, intramuscular, intrastemal injection or infusion techniques), by inhalation spray, or rectally, in dosage unit formulations containing conventional non-toxic pharmaceutically-acceptable carriers, adjuvants and vehicles.

The term “administration” and variants thereof (e.g., “administering” a compound) in reference to a potassium salt of Compound A mean providing the salt to the individual in need of treatment. When a salt of the invention is provided in combination with one or more other active agents (e.g., AIDS antivirals), “administration” and its variants are each understood to include concurrent and sequential provision of the salt and other agents.

As used herein, the term “composition” is intended to encompass a product comprising the specified ingredients in the specified amounts, as well as any product which results, directly or indirectly, from combination of the specified ingredients in the specified amounts.

The expression “pharmaceutically acceptable” means that the carrier, diluent or excipient must be compatible with the other ingredients of the formulation and not deleterious to the recipient thereof.

The term “subject,” (alternatively referred to herein as “patient”) as used herein refers to an animal, preferably a mammal, most preferably a human, who has been the object of treatment, observation or experiment.

The term “therapeutically effective amount” as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated. For the purpose of prevention of a given disease or condition, a therapeutically effective amount can alternatively be referred to as a prophylactic amount of the active compound or agent.

The pharmaceutical compositions of the present invention may be in the form of orally-administrable capsules, suspensions or tablets, or as nasal sprays, sterile injectible preparations, for example, as sterile injectible aqueous or oleagenous suspensions or suppositories. In one embodiment, the pharmaceutical composition is a capsule or a tablet suitable for oral administration comprising a potassium salt of Compound A (e.g., the crystalline K salt ethanolate) and a nonionic surfactant (e.g., a polox amer).

When administered orally as a suspension, these compositions are prepared according to techniques well-known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The injectible solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

A Compound A potassium salt of this invention can be administered orally to humans on an active ingredient basis in a dosage range of 0.01 to 1000 mg/kg body weight per day in a single dose or in divided doses. One preferred dosage range is 0.1 to 200 mg/kg body weight per day orally in a single dose or in divided doses. Another preferred dosage range is 0.5 to 100 mg/kg body weight per day orally in single or divided doses. For oral administration, the compositions are preferably provided in the form of tablets containing 1 to 1000 milligrams of the active ingredient, particularly 1, 5, 10, 15. 20, 25, 50, 75, 100, 150, 200, 250, 300, 400, 500, 600, 750, 800, 900, and 1000 milligrams of the active ingredient for the symptomatic adjustment of the dosage to the patient to be treated. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. A suitable dosage range for oral administration of a potassium salt of Compound A to humans is in a range of from about 25 mg to about 1000 mg per day (e.g., from about 100 mg to about 800 mg per patient once per day).

The present invention is also directed to combinations of a potassium salt of Compound A of the present invention with one or more agents useful in the treatment of HIV infection and/or AIDS. For example, a Compound A salt of this invention may be effectively administered, whether at periods of pre-exposure and/or post-exposure, in combination with effective amounts of the HIV/AIDS antivirals, such as those in Table 1 as follows:

| | Manufacturer | |
|---|---|---|
| Drug Name | (Tradename and/or Location) | Indication (Activity) |
| abacavir | Glaxo Welcome | HIV infection, AIDS, ARC |
| GW 1592 | (ZIAGEN ®) | (nucleoside reverse |
| 1592U89 | | transcriptase inhibitor) |
| abacavir + lamivudine + | GlaxoSmithKline | HIV infection, AIDS, ARC |
| zidovudine | (TRIZIVIR ®) | (nucleoside reverse |
| | | transcriptase inhibitors) |
| acemannan | Carrington Labs | ARC |
| | (Irving, TX) | |
| ACH 126443 | Achillion Pharm. | HIV infections, AIDS, ARC |
| | | (nucleoside reverse |
| | | transcriptase inhibitor) |
| acyclovir | Burroughs Wellcome | HIV infection, AIDS, ARC, |
| | | in combination with AZT |
| AD-439 | Tanox Biosystems | HIV infection, AIDS, ARC |
| AD-519 | Tanox Biosystems | HIV infection, AIDS, ARC |
| adefovir dipivoxil | Gilead | HIV infection, AIDS, ARC |
| GS 840 | | (reverse transcriptase |
| | | inhibitor) |
| AL-721 | Ethigen | ARC, PGL, HIV positive, |
| | (Los Angeles, CA) | AIDS |
| alpha interferon | GlaxoSmithKline | Kaposi's sarcoma, HIV, in |
| | | combination w/Retrovir |
| AMD3100 | AnorMed | HIV infection, AIDS, |
| | | ARC |
| | | (CXCR4 antagonist) |
| amprenavir | GlaxoSmithKline | HIV infection, AIDS, |
| 141 W94 | (AGENERASE ®) | ARC (PI) |
| GW 141 | | |
| VX478 (Vertex) | | |
| ansamycin | Adria Laboratories | ARC |
| LM 427 | (Dublin, OH) | |
| | Erbamont | |
| | (Stamford, CT) | |
| antibody which neutralizes | Advanced Biotherapy | AIDS, ARC |
| pH labile alpha aberrant | Concepts (Rockville, | |
| interferon | MD) | |
| AR177 | Aronex Pharm | HIV infection, AIDS, ARC |
| atazanavir (BMS 232632) | Bristol-Myers Squibb | HIV infection, AIDS, ARC |
| | (ZRIVADA ®) | (protease inhibitor) |
| beta-fluoro-ddA | Nat'l Cancer Institute | AIDS-associated diseases |
| BMS-232623 | Bristol-Myers Squibb/ | HIV infection, AIDS, |
| (CGP-73547) | Novartis | ARC |
| | | (protease inhibitor) |
| BMS-234475 | Bristol-Myers Squibb/ | HIV infection, AIDS, |
| (CGP-61755) | Novartis | ARC (protease inhibitor) |
| capravirine | Pfizer | HIV infection, AIDS, |
| (AG-1549, S-1153) | | ARC (non-nucleoside |
| | | reverse transcriptase |
| | | inhibitor) |
| CI-1012 | Warner-Lambert | HIV-1 infection |
| cidofovir | Gilead Science | CMV retinitis, herpes, |
| | | papillomavirus |
| curdlan sulfate | AJI Pharma USA | HIV infection |
| cytomegalovirus immune | MedImmune | CMV retinitis |
| globin | | |
| cytovene | Syntex | sight threatening CMV |
| ganciclovir | | peripheral CMV |
| | | retinitis |
| delavirdine | Pharmacia-Upjohn | HIV infection, AIDS, |
| | (RESCRIPTOR ®) | ARC (non-nucleoside |
| | | reverse transcriptase |
| | | inhibitor) |
| dextran Sulfate | Ueno Fine Chem. Ind. | AIDS, ARC, HIV |
| | Ltd. (Osaka, Japan) | positive asymptomatic |
| ddC | Hoffman-La Roche | HIV infection, AIDS, ARC |
| (zalcitabine, | (HIVID ®) | (nuclesodie reverse |
| dideoxycytidine) | | transcriptase inhibitor) |
| ddI | Bristol-Myers Squibb | HIV infection, AIDS, ARC; |
| dideoxyinosine | (VIDEX ®) | combination with AZT/d4T |
| | | (nucleoside reverse |
| | | transcriptase inhibitor) |
| DPC 681 & DPC 684 | DuPont | HIV infection, AIDS, ARC |
| | | (protease inhibitors) |
| DPC 961 & DPC 083 | Bristol-Myers Squibb | HIV infection AIDS, ARC |
| | (from DuPont Pharma) | (non-nucleoside reverse |
| | | transcriptase inhibitors) |
| EL10 | Elan Corp, PLC | HIV infection |
| | (Gainesville, GA) | |
| efavirenz | Bristol-Myers Squibb | HIV infection, AIDS, |
| (DMP 266) | (SUSTIVA ®) | ARC (non-nucleoside RT |
| | Merck (STOCRIN ®) | inhibitor) |
| famciclovir | Novartis | herpes zoster, herpes |
| | (FAMVIR ®) | simplex |
| emtricitabine | Gilead (from Triangle | HIV infection, AIDS, ARC |
| FTC | Pharmaceuticals) | (nucleoside reverse |
| | (COVIRACIL ®) | transcriptase inhibitor) |
| | Emory University | |
| emvirine | Gilead (from Triangle | HIV infection, AIDS, ARC |
| | Pharmaceuticals) | (non-nucleoside reverse |
| | (COACTINON ®) | transcriptase inhibitor) |
| enfuvirtide | Trimeris & Roche | HIV infection, AIDS, ARC |
| T-20 | (FUZEON ®) | (fusion inhibitor) |
| HBY097 | Hoechst Marion Roussel | HIV infection, AIDS, ARC |
| | | (non-nucleoside reverse |
| | | transcriptase inhibitor) |
| fosamprenavir | Glaxo Smith Kline | HIV infection, AIDS, ARC |
| | | (prodrug of the PI |
| | | amprenavir) |
| hypericin | VIMRx Pharm. | HIV infection, AIDS, ARC |
| recombinant human | Triton Biosciences | AIDS, Kaposi's sarcoma, |
| interferon beta | (Almeda, CA) | ARC |
| interferon alfa-n3 | Interferon Sciences | ARC, AIDS |
| indinavir | Merck (CRIXIVAN ®) | HIV infection, AIDS, ARC, |
| | | asymptomatic HIV positive, |
| | | also in combination with |
| | | AZT/ddI/ddC |
| ISIS 2922 | ISIS Pharmaceuticals | CMV retinitis |
| JE2147/AG1776 | Agouron | HIV infection, AIDS, ARC |
| | | (protease inhibitor) |
| KNI-272 | Nat'l Cancer Institute | HIV-assoc. diseases |
| lamivudine, 3TC | GlaxoSmithKline | HIV infection, AIDS, |
| | (EPIVIR ®) | ARC (nucleoside reverse |
| | | transcriptase inhibitor); |
| | | also with AZT |
| lobucavir | Bristol-Myers Squibb | CMV infection |
| lopinavir (ABT-378) | Abbott | HIV infection, AIDS, ARC |
| | | (protease inhibitor) |
| lopinavir + ritonavir | Abbott (KALETRA ®) | HIV infection, AIDS, ARC |
| (ABT-378/r) | | (protease inhibitor) |
| mozenavir | AVID (Camden, NJ) | HIV infection, AIDS, ARC |
| (DMP-450) | | (protease inhibitor) |
| nelfinavir | Agouron | HIV infection, AIDS, |
| | (VIRACEPT ®) | ARC (protease inhibitor) |
| nevirapine | Boeheringer | HIV infection, AIDS, |
| | Ingleheim | ARC (non-nucleoside |
| | (VIRAMUNE ®) | reverse transcriptase |
| | | inhibitor) |
| novapren | Novaferon Labs, Inc. | HIV inhibitor |
| | (Akron, OH) | |
| peptide T | Peninsula Labs | AIDS |
| octapeptide | (Belmont, CA) | |
| sequence | | |
| PRO 140 | Progenics | HIV infection, AIDS, ARC |
| | | (CCR5 co-receptor inhibitor) |
| PRO 542 | Progenics | HIV infection, AIDS, ARC |
| | | (attachment inhibitor) |
| trisodium | Astra Pharm. Products, | CMV retinitis, HIV infection, |
| phosphonoformate | Inc | other CMV infections |
| PNU-140690 | Pharmacia Upjohn | HIV infection, AIDS, ARC |
| | | (protease inhibitor) |
| probucol | Vyrex | HIV infection, AIDS |
| RBC-CD4 | Sheffield Med. Tech | HIV infection, AIDS, |
| | (Houston TX) | ARC |
| ritonavir | Abbott | HIV infection, AIDS, |
| (ABT-538) | (RITONAVIR ®) | ARC (protease inhibitor) |
| saquinavir | Hoffmann-LaRoche | HIV infection, AIDS, |
| | (FORTOVASE ®) | ARC (protease inhibitor) |
| stavudine; d4T | Bristol-Myers Squibb | HIV infection, AIDS, ARC |
| didehydrodeoxy- | (ZERIT ®) | (nucleoside reverse |
| thymidine | | transcriptase inhibitor) |
| T-1249 | Trimeris | HIV infection, AIDS, ARC |
| | | (fusion inhibitor) |
| TAK-779 | Takeda | HIV infection, AIDS, ARC |
| | | (injectable CCR5 receptor |
| | | antagonist) |
| tenofovir | Gilead (VIREAD ®) | HIV infection, AIDS, ARC |
| | | (nucleotide reverse |
| | | transcriptase inhibitor) |
| tipranavir (PNU-140690) | Boehringer Ingelheim | HIV infection, AIDS, ARC |
| | | (protease inhibitor) |
| TMC-120 & TMC-125 | Tibotec | HIV infections, AIDS, ARC |
| | | (non-nucleoside reverse |
| | | transcriptase inhibitors) |
| TMC-126 | Tibotec | HIV infection, AIDS, ARC |
| | | (protease inhibitor) |
| valaciclovir | GlaxoSmithKline | genital HSV & CMV |
| | | infections |
| virazole | Viratek/ICN (Costa | asymptomatic HIV positive, |
| ribavirin | Mesa, CA) | LAS, ARC |
| zidovudine; AZT | GlaxoSmithKline | HIV infection, AIDS, ARC, |
| | (RETROVIR ®) | Kaposi's sarcoma in |
| | | combination with other |
| | | therapies (nucleoside reverse |
| | | transcriptase inhibitor) |

Other agents suitable for administration with the Compound A salt of this invention include those set forth in the Table of antivirals, immuno-modulators, anti-infectives, and other agents in WO 02/30930, the disclosure of which is herein incorporated by reference in its entirety. It will be understood that the scope of combinations of a Compound A salt of this invention with HIV/AIDS antivirals, immunomodulators, anti-infectives or vaccines is not limited to the list in Table 1 above and in the Table in WO 02/30930, but includes in principle any combination with any pharmaceutical composition useful for the treatment of HIV infection and/or AIDS. When employed in combination with a salt of the invention, the HIV/AIDS antivirals and other agents are typically employed in their conventional dosage ranges and regimens as reported in the art, including the dosages described in the Physicians' Desk Reference, 54^{th} edition, Medical Economics Company, 2000. The dosage ranges for a compound of the invention in these combinations are the same as those set forth above just before Table 1.

One suitable combination is a potassium salt of Compound A of the present invention and a nucleoside inhibitor of HIV reverse transcriptase such as AZT or 3TC, ddC, or ddl. Another suitable combination is a Compound A salt of the present invention and a non-nucleoside inhibitor of HIV reverse transcriptase, such as efavirenz, and optionally a nucleoside inhibitor of HIV reverse transcriptase, such as AZT, 3TC, ddC or ddl.

In the above-described combinations, a Compound A potassium salt of the present invention and other active agents may be administered together or separately. In addition, the administration of one agent may be prior to, concurrent with, or subsequent to the administration of other agent(s). These combinations may have unexpected or synergistic effects on limiting the spread and degree of infection of HIV.

Abbreviations used herein include the following:
AIDS=acquired immunodeficiency syndrome
ARC=AIDS related complex
Bn=benzyl
BOC or Boc=t-butyloxycarbonyl
Bu=butyl
DMF=N,N-dimethylformamide
DSC=differential scanning calorimetry
DIPA=diisopropylamine
EDC=1-ethyl-3-(3-dimethylaminopropyl) carbodiimide
EDTA=ethylenediamine tetraacetic acid
EtOAc=ethyl acetate
EtOH=ethanol
g=gram(s)
h=hour(s)
HIV=human immunodeficiency virus
HOBT or HOBt=1-hydroxy benzotriazole hydrate
HPLC=high-performance liquid chromatography
IPAc=isopropyl acetate
Me=methyl
MeCN=acetonitrile
MeOH=methanol
min=minute(s)
Ms=mesyl (methanesulfonyl)
MIBE=methyl t-butyl ether
NMM=N-methylmorpholine
NMR=nuclear magnetic resonance
TEA=triethylamine
THF=tetrahydrofuran
Ts=tosyl or toluenesulfonyl
XRPD=x-ray powder diffraction

The following examples serve only to illustrate the invention and its practice. The examples are not to be construed as limitations on the scope or spirit of the invention.

### EXAMPLE 1

Preparation of 1,4-Butanesultam

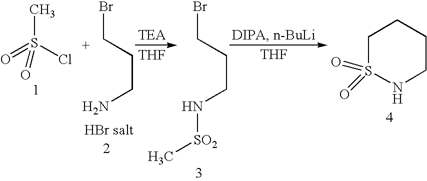

| | | | | E- | | |
|---|---|---|---|---|---|---|
| | Weight | FW | Moles | quiv. | Density | Volume |
| MsCl (1) | 2.36 Kg | 114.55 | 20.6 | 1.03 | 1.480 | 1.59 L |
| 3-bromo- | 4.40 Kg | 220 | 20.0 | 1.00 | | |
| propyl-amine | | | | | | |
| (2) HBr salt | | | | | | |
| TEA | 4.07 Kg | 101.19 | 40.2 | 2.01 | 0.726 | 5.60 L |
| THF | | | | | 43 + 4 + 8 = 55 L | |
| DIPA | 481 g | 101.19 | 4.75 | 0.25 | 0.722 | 666 mL |
| 1,10-Phenan- | 4.11 g | 180.21 | | | | |
| throline | | | | | | |
| n-BuLi, 1.6 M in | | | | | | |
| hexane | | | | | | |

The 3-bromopropylamine-HBr salt (2) and THF (43 L) were placed in a 72 L round-bottomed-flask under N₂ and the resulting slurry was cooled to 0° C. Two dropping funnels were fitted to the flask. One was charged with the TEA and the other with a solution of the MsCl (1) and THF (4L). The contents of the addition funnels were added at roughly the same rate (the TEA was added slightly faster than the MsCl) while maintaining an internal reaction temperature below 10° C. The addition required 2 h. The resulting white suspension was warmed to 23° C. and aged for 1 h. The suspended solids (a mixture of TEA-HBr and TEA-HCl) were removed by filtration through a dry frit. The cake was washed with THF (8L). The combined filtrate and cake-rinse, a THF solution of 3, was collected in a 100 L round-bottomed-flask under N₂. To the solution of 3 was added the 1,10-phenanthroline and the DIPA and the resulting solution was cooled to −30° C. The n-BuLi was added over about 4 h maintaining the internal temperature below −20° C. After 1.25 eq of the n-BuLi was added the reaction mixture became deep brown and the color remained as the addition was completed. The reaction mixture was warmed to 0° C. over 3 h. A small aliquot was removed, and partitioned between saturated NH₄Cl and EtOAc. The EtOAc was evaporated and the residue examined by ¹H NMR to confirm consumption of 3 and conversion to 4. To the reaction mixture at 0° C. was added saturated aqueous NH₄Cl (12 L, the first 1 L slowly, a heat kick to 6° C. was observed) and then brine (12 L). The phases were partitioned and the aqueous phase was extracted with EtOAc (20 L). The organic phases were combined, washed with brine (4 L) and then concentrated under vacuum to about 12 L. The solvent was switched to EtOAc (20 L used) maintaining a volume of 12 L. After the solvent switch, a yellow slurry resulted. n-Heptane (20 L) was added with stirring and the slurry was cooled to 5° C. After a 1 h age the solids were collected on a frit and rinsed with cold (5° C.) 3:5. EtOAc/n-heptane. The wet cake was dried for 24 h under a stream of dry N₂ to provide 1.44 Kg (53% from 2) of sultam 4 as a crystalline yellow solid.

¹H NMR (CDCl₃, 400 ml) δ 4.36 (br s, 1H), 3.45 (m, 2H), 3.10 (m, 2H), 2.24 (m, 2H), 1.64 (m, 2H).

### EXAMPLE 2

Alternative Preparation of 1,4-Butanesultam
Step 1:

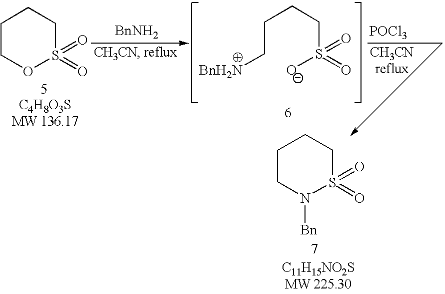

| Materials | MW | Amount | | Moles | Equivalent |
|---|---|---|---|---|---|
| 1,4-Butane sultone | 136.17 | 68.10 | g | 0.5000 | 1 |
| Benzylamine | 107.16 | 69.70 | g | 0.6500 | 1.3 |
| Acetonitrile | | 625 | mL | | |
| Phosphorus oxychloride | 153.33 | 153.33 | g | 1.000 | 2 |

A solution of 1,4-butane sultone 5 (68.10 g, 0.5000 moles) and benzylamine (69.70 g, 0.6500 moles) in acetonitrile (625 mL) was refluxed at 82° C. for 24 hours, with the reaction monitored by ¹H NMR until conversion of 5 to 6 was >98%. While the resulting slurry was cooled to 50° C., phosphorus oxychloride (153.33 g, 1.000 moles) was slowly added via a dropping funnel. After complete addition, the mixture was refluxed at 82° C. for 8 hours, with the reaction monitored by HPLC until conversion was >98%. The reaction mixture was concentrated to remove acetonitrile, and the residue was cooled to 0-5° C. and neutralized with 20% sodium hydroxide to pH=7. The resulting mixture was extracted with IPAc (3×350 mL), and the combined extracts were washed with 10% sodium bicarbonate (2×100 mL) and 25% of brine (100 mL). The resulting clear solution was concentrated and solvent switched to methanol (total volume 1000 mL), which was used in the next step of the reaction. For compound 7: ¹H NMR (CDCl₃, 400 MHz) δ: 7.38-7.32 (m, 5 H), 4.32 (s, 2H), 3.23 (m, 2 H), 3.11 (m, 2 H), 2.22 (m, 2 H), 1.62 (m, 2 H).

Step 2:

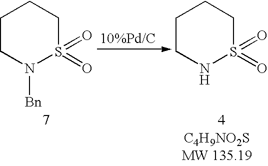

| Materials | MW | Amount | | Moles | Equivalent |
|---|---|---|---|---|---|
| N-Benzyl-1,4-butanesultam | 225.30 | | | 0.5000 | 1 |
| 10% Pd/C | | 12.0 | g | | 10% wt |
| 1 N HCl (aqueous) | | 80 | mL | | |
| Solka Flock | | 20 | g | | |

To a solution of N-Benzyl-1,4-butanesultam 7 (0.5000 moles) in methanol (total volume 1000 mL) and 1 N HCl aqueous (80 mL) was added 10% Pd/C (12.0 g). The resulting slurry was submitted to hydrogenation at 40° C., 45 psi for 24 hours, with the reaction monitored by HPLC until conversion of 7 to 4 was >99%. The reaction mixture was cooled to ambient temperature and filtered by passing through a pad of Solka Flock (20 g) and washed with methanol (3×100 mL). The combined filtrates were concentrated to remove the methanol, and a crystalline solid was precipitated out during the concentration. To the slurry solution was added heptane/MTBE (3:2, 100 mL). The resulting mixture was cooled to 0° C., and aged for 0.5 hour. The crystalline solid was filtered off and washed with cold heptane/MTBE (3:2, 50 mL), and dried under vacuum with a nitrogen sweep to give 1,4-butanesultam 4 (49.8 g, 74% overall from 5).

### EXAMPLE 3

### Preparation of 5-(1,1-dioxido-1,2-thiazinan-2-yl)-8-(4-toluenesulfonyloxy)-1,6-naphthyridine-7-carboxylic acid methyl ester

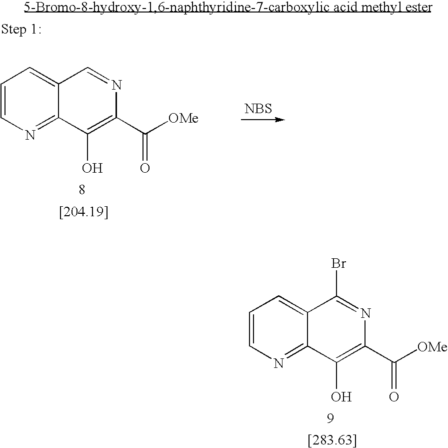

N-bromosuccinimide (7.83 g, 44.0 mmol) was added to a solution of 8-hydroxy-1,6-naphthyridine-7-carboxylic acid methyl ester (8, 8.17 g, 40.0 mmol) in chloroform (32 mL) over 20 min maintaining the temperature at 20-50° C. and the mixture was aged for 30 min at 50° C. The mixture became a thick, stirrable slurry and HPLC analysis indicated <2% starting material remaining. The mixture was cooled to 30° C. over 15 min. MeOH (64 mL) was added over 30 min then a 1:1 mixture of MeOH-water (64 mL) was added over 30 min. The mixture was cooled to −40° C. over 30 min and aged at −40° C. for 30 min. The cold mixture was filtered and the solid was washed with 1:1 MeOH:water (100 mL) at 10-20° C. The off white crystalline solid was dried under a stream of nitrogen to provide 10.48 g (93% yield) of 5-bromo-8-hydroxy-1,6-naphthyridine-7-carboxylic acid methyl ester (9).

HPLC retention times: 8=2.2 min, 2=6.0 min, HPLC conditions: 150×4.6 mm ACE 3 C18 column, isocratic elution with 30% MeCN in 0.025% aq H₃PO₄ at 1 mLjmin, 25° C. with detection at 254 nm;

HPLC retention times: 8=1.8 min, 2=3.1 min, HPLC conditions: 150×4.6 mm ACE 3 C18 column, isocratic elution with 46% MeCN in 0.025% aq H₃PO₄ at 1 mLjmin, 25° C. with detection at 254 nm.

¹³C NMR of 9 (CDCl₃, 100 MHz): 169.7, 156.3, 154.5, 143.9, 137.1, 132.4, 128.0, 126.1, 124.2, 53.4.

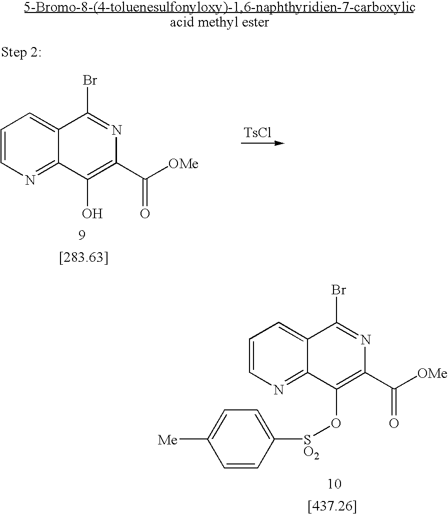

Triethylamine (0.759 g, 7.50 mmol) was added to a suspension of 5-bromo-8-hydroxy-1,6-naphthyridine-7-carboxylic acid methyl ester (2, 1.415 g, 5.000 mmol) in chloroform (5 mL) over 5 min maintaining the temperature at 20-50° C. to give a yellow suspension. p-Toluenesulfonyl chloride (1.15 g, 6.00 mmol) was added over 5 min maintaining the temperature at 20-40° C. to give a yellow solution. The mixture was aged at 40° C. for 2 h during which a crystalline solid precipitated out of the mixture and the color faded (HPLC analysis indicated <0.5% starting material remaining). The mixture was cooled to 20° C. over 15 min. MeOH (10 mL) was added over 30 min then a 1:1 mixture of MeOH:water (10 mL) was added over 30 min. The mixture was cooled to 40° C. over 30 min and aged at −40° C. for 30 min. The cold mixture was filtered and the solid was washed with 1:1 MeOH:water (10 mL), MeOH (5 mL), MIBE (10 mL) and hexanes (10 mL) all at 10-20° C. The off-white crystalline solid was dried under a stream of nitrogen to provide 2.112 g (97% yield) of 5-bromo-8-p-toluenesulfonyloxy)-1,6-naphthyridine-7-carboxylic acid methyl ester (10).

HPLC retention times: 9=3.1 min, 10=12.4 min, HPLC conditions: 150×4.6 mm ACE 3 C18 column, isocratic elution with 46% MeCN in 0.025% aq H₃PO₄ at 1 mL/min, 25° C. with detection at 254 nm.

¹³C NMR of 10 (d6-DMSO, 100 MHz): 163.2, 157.0, 146.5, 145.8, 141.9, 141.3, 139.2, 137.2, 132.3, 130.4, 129.0, 127.6, 127.1, 53.3, 21.7.

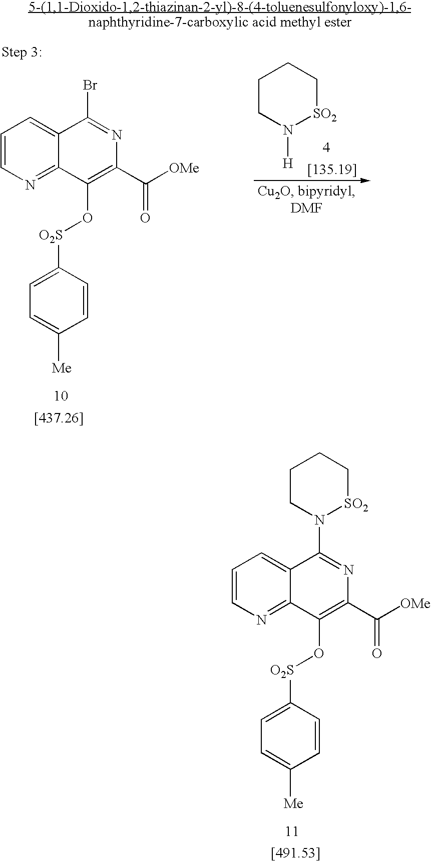

A mixture of 5-bromo-8-(p-toluenesulfonyloxy)-1,6-naphthyridine-7-carboxylic acid methyl ester (10, 2.186 g, 5.000 mmol), 1,4-butane sultam (4, 811 mg, 6.00 mmol), copper (I) oxide (858 mg, 6.00 mmol, <5 micron), 2,2′-bipyridyl (937 mg, 6.00 mmol) and DMF (10 mL) was degassed by stirring under a stream of nitrogen for 1 min and heated to 120° C. for 4 h. The brown suspension became a dark red solution with a small amount of undissolved copper (I) oxide remaining (HPLC analysis indicated <0.5% starting material remaining). The mixture was diluted with chloroform (10 mL), Solkaflok (200 mg) was added and the resulting mixture was filtered through a plug of Solkaflok. The plug was washed with chloroform (10 mL) and the combined filtrates were stirred vigorously with a solution of EDTA disodium salt dihydrate (3.8 g, 10.2 mmol) in water (40 mL) while air was slowly bubbled in for 40 min. The upper aqueous phase became turquoise while the lower organic phase became yellow. The organic phase was washed with a solution of EDTA disodium salt (1.9 g, 5.1 mmol) in water (30 mL) and a solution of sodium bisulfate monohydrate (0.87g, 6.3 mmol) in water (30 mL). Each of the above three aqueous phases was back extracted sequentially with one portion of chloroform (15 mL). The organic phases were dried over sodium sulfate and filtered. The dried organic extracts were concentrated and solvent switched to a final volume of 15 mL MeOH using a total of 30 mL MeOH for the switch at atmospheric pressure. Product crystallized during the solvent switch. The resulting slurry was cooled to 0° C. over 30 min and aged at 0° C. for 30 min. The slurry was filtered cold and the solid was washed with MeOH (15 mL). The off white solid was dried under a stream of nitrogen to provide 1.910 g (78%) of 5-(N-1,4-butanesultam)-8-(p-toluenesulfonyloxy)-1,6-naphthyridine-7-carboxylic acid methyl ester (11).

HPLC retention times: 10=12.4 min, 11=10.3 min, DMF=1.3 min, Bipy=1.5 min, HPLC conditions: 150×4.6 mm ACE 3 C18 column, isocratic elution with 46% MeCN in 0.025% aq H₃PO₄ at 1 mL/min, 25° C. with detection at 254 nm.

¹³C NMR of 11 (CDCl₃, 100 MHz): 164.2, 155.3, 151.9, 146.7, 145.4, 141.2, 137.8, 135.3, 133.6, 129.6, 128.9, 125.4, 124.3, 53.4, 52.9, 48.7, 24.2, 22.0, 21.7.

### EXAMPLE 4

### Potassium 5-(1,1 -dioxido-1,2-thiazinan-2-yl)-7-[({4-fluoro-2-[(methylamino)-carbonyl]benzyl}amino)carbonyl]-1,6-naphthyridin-8-olate

Step 1: Methyl 2-bromo-5-fluorobenzoate

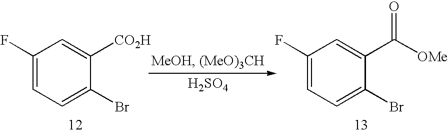

| Material | MW | Amount | | Moles |
|---|---|---|---|---|
| 2-bromo-5-fluorobenzoic | 219.01 | 4.00 | g | 18.3 |
| acid | | | | |
| methanol | 32.04 | 18 | L | 296.3 |
| | (d = 0.791) | | | |
| trimethylorthoformate | 106.12 | 3.88 | kg | 36.5 |
| 96% sulfuric acid | 98.08 | 0.373 | kg | 3.65 |
| 2 M K₂HPO₄ | 174.18 | 4.82 | L | 9.68 |
| ethyl acetate | | 16 | L | |
| 10% NaHCO₃ | 84.02 | 4 | L | |
| 25% brine | | 4 | L | |
| toluene | | 12 | L | |
| DMF | | | | |

To a 72 L round bottom flask, equipped with an overhead stirrer, thermocouple, water-cooled condenser, and nitrogen inlet, was charged methanol (18 L). 2-Bromo-5-fluorobenzoic acid (4.00 kg), trimethyl orthoformate (3.876 kg), were then charged with stirring, followed by the addition 96% sulfuric acid (0.373 kg). The resulting solution was refluxed at 63° C. and aged for 10-16 hr, while the by-product (methyl formate) was removed during the reaction. The reaction mixture was monitored by TPLC (conversion was >99%). The reaction mixture was concentrated, then diluted with ethyl acetate (16 L), and cooled to 20° C. 2 M potassium hydrogen phosphate (4.82 L) was then added to adjust the pH to 6.5-7. The mixture was then transferred to a 100 L nalgene extractor. After phase cut, the organic layer was washed with 10% NaHCO₃ (4 L), 25% brine (4 L), and then concentrated under reduced pressure. The residual oil was dissolved in toluene (6 L), and concentrated. This operation was done one more time. The remaining oil was dissolved in DMF (total vol. 9.2 L). The resulting solution was used for next step.

HPLC conditions: column: Zorbax, Rx C8 250×4.6 mm; temperature: 30° C; detection: 210 nm; mobile phase: 0.1% aq H₃PO₄ (A)/MeCN (B); gradient: 90:10 (A)/(B) to 10:90 over 15 min, 10:90 hold for 5 min, 10:90 to 90:10 (A)/(B) over 10 seconds; flow rate: 1 mL/min; retention time for the desired monoester; 13.6 min.

Evaporation of a sample to dryness gave a colorless oil: ¹H NMR (400 MHz, CDCl₃) δ: 7.64 (dd, J=8.8, 5.0 Hz, 1H), 7.53 (dd, J=8.8, 3.1 Hz, 1 H), 7.08 (td, J=8.8, 3.1 Hz, 1 H), 3.95 (s, 3H); ¹³C NoM (100 MHz, CDCl₃) δ: 165.4, 161.3 (d, J=240.0 Hz), 135.9, 133.4, 120.0 (d, J=20.0 Hz), 118.5 (d, J=20.0 Hz), 116.1, 52.7.

Step 2: Methyl 2-nitrile-5-fluorobenzoate

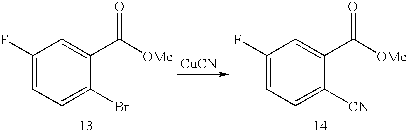

| Material | MW | Amount | Moles |
|---|---|---|---|
| methyl 2-bromo-5-fluoro- | 233.03 | | 18.3 in DMF |
| benzoate | | | |
| copper(I) cyanide | 89.56 | 1.60 kg | 17.9 |
| DMF | | 5 L + 4 L | |
| ethyl acetate | | 35 L + 17 L | |
| 10% NH₄OH-20% NH₄Cl | | 37 L | |
| 25% brine | | 8 L | |
| MeOH | | 33 L | |

To a solution of methyl 2-bromo-5-fluorobenzoate (18.26 moles) in DMF (total vol. 9.2 L) was charged copper(I) cyanide (1.603 kg) in DMF (5 L) slurry and followed with a DMF flush (4 L). After being degassed, the reaction mixture was heated at 100° C. for 10-16 hours. The reaction mixture was monitored by HPLC (conversion was >98%). After being cooled to 50° C.-60° C., ethyl acetate (20 L) was added, and then 10% NH4OH-20% NH₄Cl (22 L). The mixture was then transferred to a 100 L nalgene extractor. The 72 L round bottom flask was washed with 15 L of EtOAc and 15 L of water and transferred to the 100 L extractor. After phase cut, the aqueous layer was back-extracted with EtOAc (17 L) one time. The combined organic layers were washed with 10% NH₄OH/20% NHCl: water (1:1, 3×10 L), 16% brine (8 L), concentrated, and solvent switched to MeOH (total vol. 22 L, KF=152.6 μg/mL). The resulting solution was used for next step.

HPLC conditions: column: Zorbax, Rx C8 250×4.6 mm; temperature: 30° C; detection at 210 nm; mobile phase: 0.1% aq H₃PO₄ (A)/MeCN (B); gradient: 90:10 (A)/(B) to 10:90 over 15 min, 10:90 hold for 5 min, 10:90 to 90:10 (A)/(B) over 10 seconds; flow rate: 1 mL/min; retention time for the desired monoester: 11.7 min.

Evaporation of a sample to dryness gave a light yellow solid: ¹H NMR (CDCl₃) δ: 7.86-7.80 (m, 2 H), 7.37 (td, J=8.5, 2.6 H, 1 H), 4.02 (s, 3 H); ¹³C NMR (100 MHz, CDCl₃) δ: 164.3 (d, J=260 Hz), 163.3, 137.1 (d, J=10.0 Hz), 135.2 (d, J=10.0 HZ), 120.2 (d, J=30.0 Hz), 118.8 (d, J=20.0 Hz), 116.6, 109.0, 53.1.

Step 3: Methyl 2-aminomethyl-5-fluorobenzoate, HCl salt

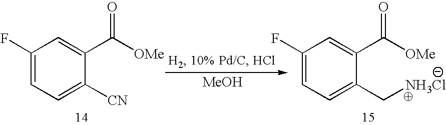

| Material | MW | Amount | Moles |
|---|---|---|---|
| methyl 2-nitrile-5-fluoro- | 179.15 | | 10.6 in MeOH |
| benzoate | | | |
| 3.0 M HCl in MeOH | 36.46 | 7.10 L | 21.22 |
| (anhydrous) | | | |
| 10% Pd/C | | 0.475 kg | |
| solka floc | | 2.6 kg | |
| MeOH | | 3 × 10 L | |

A degassed mixture of methyl 2-nitrile-5-fluorobenzoate (10.6 moles) in MeOH (total 10.0 L), 3.0 M HCl in MeOH (7.10 L), and 10% Pd/C (0.475 kg) was submitted to hydrogenation at 40° C. and 45 PSI for 48 hours. The reaction mixture was monitored by HPLC (conversion was >97%). After being cooled to ambient temperature, the reaction mixture was then filtered by passing a short Solka Flock (2.6 kg), which was washed with MeOH (3×10 L). The combined filtrates were concentrated and solvent-switched to toluene in total volume (about 18 L, KF=154 μg/mL). The crystalline solid was filtered off and washed with toluene, dried under vacuum with nitrogen sweep to afford 2.02 kg of the title compound (87% isolated yield overall for the three steps, >99A % purity, HPLC).

HPLC conditions: column: Zorbax, Rx C8 250×4.6 mm; temperature: 30° C; detection at 210 nm; mobile phase: 0.1% aq H₃PO₄ (A)/MeCN (B); gradient: 90:10 (A)/(B) to 10:90 over 15 min, 10:90 hold for 5 min, 10:90 to 90:10 (A)/(B) over 10 seconds; flow rate: 1 mL/min; retention time for the desired monoester: 5.78 min.

¹H NMR (CDCl₃) δ: 8.43 (brs, 3 H), 7.74-7.65 (m, 2H), 7.55 (td, J=8.4, 2.8 Hz, 1 H), 4.26 (q, J=5.5 Hz), 3.85 (s, 3 H); ¹³C NMR (100 MHz, CDCl₃) δ: 165.8, 162.1 (d, J=250 Hz), 134.8 (d, J=10.0 Hz), 131.9 (d, J=10.0 Hz), 131.7, 120.1 (d, J=20.0 Hz), 117.7 (d, J=30.0 Hz), 53.2, 40.3.

Step 4: Methyl 2-t-butyloxycarbonylaminomethyl-5-fluorobenzoate

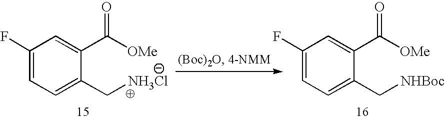

| Material | MW | Amount | | Moles |
|---|---|---|---|---|
| ammonium salt 15 | 219.64 | 3.42 | kg | 15.6 |
| (BOC)₂O | 218.25 | 3.73 | kg | 17.1 |
| NMM | 101.15 | 1.73 | kg | 17.1 |
| | (d = 0.920) | | | |
| 40 wt. % MeNH₂ | 31.06 | 1.21 | kg | 15.6 |
| toluene | | 31 | L | |
| 0.1 M EDTA Na sol'n | | 6.2 | L | |
| 25% brine | | 6.2 | L | |

To the ammonium salt 15 (3.42 kg) in toluene (31L) was added (BOC)₂O (3.73 kg), followed by NMM (1.73 kg), at 15° C.-20° C. over 1 hour. The reaction mixture was aged at room temperature for 15-24 hours (conversion as determined by HPLC was >99%), followed by the addition of 40 wt % methylamine aqueous (1.21 kg) at 5° C.-10° C., after which the mixture was aged at the same temperature for 2 hours to quench the excess (BOC)₂O. The reaction mixture was then worked up by charging water (12 L). After phase cut, the organic layer was washed with 0.1 M EDTA sodium solution (6.2 L), 25% brine (6.2 L), and concentrated to total volume (20 L), which was divided by two equal amount portions for amidation in two batches.

HPLC conditions: column: Zorbax, Rx C8 250×4.6 mm; temperature: 30° C.; detection at 210 nm; mobile phase: 0.1% aq H₃PO₄ (A)/MeCN (B); gradient: 90:10 (A)/(B) to 10:90 over 15 min, 10:90 hold for 5 min, 10:90 to 90:10 (A)/(B) over 10 seconds; flow rate: 1 mL/min; retention time for the desired monoester: 14.5 min.

Evaporation of a sample to dryness gave a colorless oil: ¹H NMR (CDCl₃) δ: 7.65 (dd, J=9.4, 2.4, 1 H), 7.50 (dd, J=8.0, 5.7 Hz, 1 H), 7.18 (dd, J=8.0, 2.8 Hz, 1 H), 5.31 (brs, 1 H), 4.47 (d, J=6.6 Hz, 1 H), 3.91 (s, 3 H), 1.41 (s, 9 H); ¹³C NMR (100 MHz, CDCl₃) δ: 166.5, 1.61.5 (d, J=250 Hz), 155.8, 137.0, 132.8 (d, J=10.0 Hz), 130.2 (d, J=10.0 Hz), 119.6 (d, J=30.0 Hz), 117.7 (d, J=20.0 Hz), 79.2, 52.4, 42.9, 28.4 (3C).

Step 5: N-methyl 2-t-butyloxycarbonylaminomethyl-5-fluoro-
benzenecarboxamide

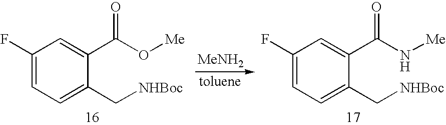

| Material | MW | Amount | Moles |
|---|---|---|---|
| methyl benzoate 16 | 283.30 | | 7.77 in toluene |
| methylamine | 31.06 | 0.483 kg | 15.6 |
| toluene | | 5 L | |
| heptane | | 50 L + 25 L | |

The crude methyl benzoate 16 in toluene (7.77 moles in 10 L) was cooled to −20° C. and methylamine (0.483 kg) gas was added. The mixture was then heated in an autoclave at 80-85° C. for 48 hours. The reaction was monitored by HPLC (conversion was >98%). After cooling to about 50° C., the reaction mixture was transferred to a large round bottom flask for batch concentration. The solution was concentrated, producing a slurry, and solvent-switched to toluene (total vol. 12 L), after which heptane (50 L) was slowly charged to the slurry. The resulting slurry was aged at 0° C. for 1 hour. The white crystalline solid was filtered off, rinsed with heptane (25 L), and dried under vacuum with a nitrogen sweep to give methylamide 17 (1.92 kg, 83% overall yield for the two preceding steps after correcting to pure product).

HPLC conditions: column: Zorbax, Rx C8 250×4.6 mm; temperature: 30° C; detection at 210 nm; mobile phase: 0.1% aq H₃PO₄ (A)/MeCN (B); gradient: 90:10 (A)/(B) to 10:90 over 15 min, 10.90 hold for 5 min, 10.90 to 90:10 (A)/(B) over 10 seconds; flow rate: 1 mL/min; retention time for the desired product: 11.6 min.

¹H NMR (CDCl₃) δ: 7.43 (dd, J=8.4, 5.5 Hz, 1 H), 7.15-7.07 (m, 2 H), 6.52 (brs, 1 H), 5.66 (brs, 1 H), 4.28 (d, J=6.4 Hz, 2 H), 3.10 (d, J=4.8 H, 3 H), 1.42 (s, 9 H); ¹³C NMR (100 MHz, CDCl₃) δ: 169.0, 161.5 (d, J=250 Hz), 156.1, 15 137.3, 133.5, 132.0 (d, J=10.0 Hz), 117.2 (, d, J=20.0 Hz), 114.3 (d, J=20.0 Hz), 79.4, 42.2, 26.7.

Step 6: N-methyl 2-amino-5-fluorobenzenecarboxamide, HCl salt

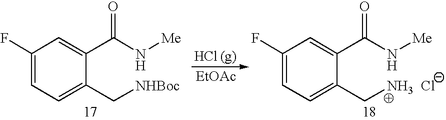

| Material | MW | Amount | Moles |
|---|---|---|---|
| N-methyl amide 17 | 282.31 | 3.14 kg | 11.1 |
| HCl (gas) | 36.46 | 3.25 kg | 89.0 |
| EtOAc | | 21.4 L + 42.8 L + 30 L | |
| heptane | | 40 L | |

HCl gas (3.25 Kg) was bubbled into ethyl acetate (21.4 L) at −20° C. N-methyl amide 17 (3.14 kg) was charged to the HCl-EtOAc solution, and the reaction mixture was warmed to ambient temperature (17° C.) in about 3 hours and aged for 2-4 hours. The reaction was monitored by HPLC (conversion was >99%). The reaction mixture was diluted with EtOAc (42.8 L), and the resulting slurry was aged at 0-5° C. for 0.5 hour. The crystalline solid was filtered off and washed with EtOAc (30 L), then with heptane (40 L), and then dried under vacuum with a nitrogen sweep to give the salt. The crystalline solid (2.434 kg) was recrystallized by dissolved in methanol (10.5 L) at 30° C. To the resulting solution was added EtOAc (64 L), producing a slurry that was aged at 0-5° C. for 1 hour. The white crystalline solid was filtered off and washed with EtOAc (30 L), dried under vacuum with nitrogen sweep to give the desired product (2.14 kg, 91% isolated yield corrected for starting material purity; >99.5 A % purity).

HPLC conditions: column: Zorbax, Rx C8 250×4.6 mm; temperature: 30° C; detection at 210 nm; mobile phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; flow rate: 1 mL/min; retention time for the desired product: 3.33 min.

¹H NMR (CDCl₃) δ: 8.84 (brs, 1 H), 8.05 (brs, 3 H), 7.55 (dd, J=8.3, 5.8 Hz, 1 H), 7.46-7.13 (m, 2 H), 4.01 (s, 3 H), 2.77 (d, J=4.6 Hz, 3 H); ¹³C NMR (100 MHz, CDCl₃) δ: 167.9, 162.0 (d, J=250 Hz), 157.9, 138.5 (d, J=10.0 Hz), 134.3 (d, J=10.0 Hz), 129.2, 117.6 (d, J=20.0 Hz), 115.5 (d, J=20.0 Hz), 40.7, 26.7.

Step 7: 5-(1,1-Dioxido-1,2-thiazinan-2-yl)-8-hydroxy-1,6-naphthy-
ridine-7-carboxylic acid

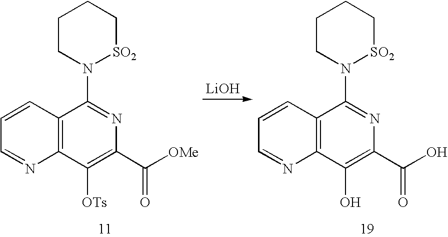

| Material | MW | Equivalents | Amount | Moles |
|---|---|---|---|---|
| Tosylate 11 | 491.5 | 1.0 | 3.3 kg | 6.7 |
| 2-propanol | | 4 L/kg 11 | 13.2 L | |
| water | | 4 L/kg 11 | 13.2 L | |
| LiOH.H₂O | 41.96 | 3.3 | 0.93 | 22.2 |
| 2 N HCl | | 2.6 | 8.7 L | 17.5 |
| Water | | 5 L/kg 11 | 4 × 4.3 L | |

A 50-L flask equipped with a mechanical stirrer, temperature probe, addition funnel, and nitrogen inlet was charged with 2-propanol (13.2 L) and tosylate 8 (3.3 kg). The lithium hydroxide monohydrate (0.93 kg) was then charged as a solution in GMP water (13.2 L) at 20-25° C. The resulting suspension was warmed to 60° C. where a homogeneous yellow solution was obtained. The reaction was aged until complete conversion to 19 was reached as determined by HPLC assay (4-16 hours). The resulting yellow suspension was cooled to about 20° C. and diluted with 2 N HCl (8.7 L) over 0.5 hour. The pH was between 1.3-1.6 at 20° C. following HCl addition. The suspension was cooled to about 20° C., filtered, and the cake was washed with water (4×4.3 L) as displacement washes. The cake was dried on the filter pot under nitrogen and house vacuum until the water content was <6 wt % by Karl Fisher titration. The purity of carboxylic acid phenol 19 was >99.4 A % by HPLC assay.

¹H NMR (DMSO-d6, 400 MHz) δ 9.21 (1H, dd, J=4.3, 1.6 Hz), 8.62 (1H, dd, J=8.5, 1.6 Hz), 7.92 (1H, dd, J=8.5, 4.3 Hz), 3.91-3.78 (2H, m), 3.55-3.45 (2H, m), 2.28 (3H, m) and 1.64 (1H, m) ppm.

Step 8: 5-(1,1-Dioxido-1,2-thiazinan-2-yl)-N-{4-fluoro-2-[(meth-
ylamino)carbonyl]benzyl}-8-hydroxy-1,6-naphthyridine-7-carbox-
amide

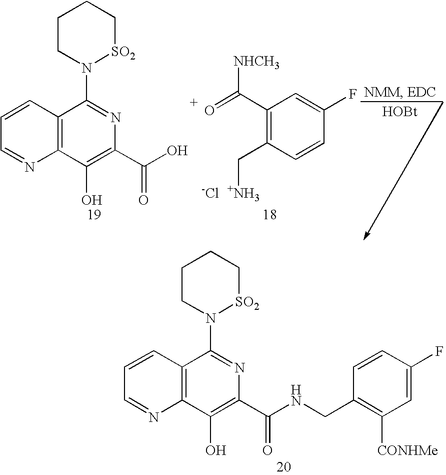

| Material | MW | Equivalents | Amount | | Moles |
|---|---|---|---|---|---|
| carboxylic acid 19 | 323.33 | 1.0 | 1.63 | kg | 5.04 |
| DMF | | 10 L/kg 19 | 16.3 | L | |
| amine 7 | 218.66 | 1.2 | 1.32 | kg | 6.05 |
| HOBt | 135.13 | 0.5 | 341 | g | 2.52 |
| NMM | 101.15 | 0.9 | 456 | g | 4.54 |
| EDC.HCl | 191.71 | 1.5 | 1.45 | kg | 7.56 |
| water | | 10 L/kg 19 | 16.3 | L | |

A 50-L flask equipped with a mechanical stirrer, temperature probe, and nitrogen inlet was charged with the dry DMF (16.3 L), carboxylic acid 19 (1.73 kg gross, 1.63 assay kg, KF=6.0 wt % water), anhydrous HOBt (341 g), amine 18 (1.32 kg), and NMM (456 g, 500 mL). The suspension was agitated at 20° C. until a homogeneous solution was obtained and then cooled to 0-5° C. The EDC (1.45 kg) was added and the reaction aged until complete conversion of 19 was reached as determined by HPLC (<0.5% 19, about 16 hours). The reaction was diluted with water (1.6 L) at 20° C., seeded (11 g), and aged for 0.5 hour. The batch was diluted with water (14.7 L) to give a 1:1 v/v ratio of water:DMF and then cooled to 0° C. The batch was then filtered and the cake washed with chilled 1:1 water:DMF (4×2.5 L) and chilled water (4×5.5 L) as displacement washes. The cake was then dried at ambient temperature under nitrogen tent/house vacuum to obtain the title product (2.16 kg, 88% isolated yield, purity: >99.0 A % by HPLC assay).

¹H NMR (DMSO-d6, 400 MHz) δ 9.53 (1H, s), 9.19 (1H, s), 8.68 (1H, s), 8.58 (1H, d, J=8.0 Hz), 7.89 (1H, d, J=3.8 Hz), 7.53 (1H, m), 7.41-7.34 (2H, m), 4.64 (2H, d, J=5.7 Hz), 3.92-3.47 (4H, m), 2.83 (3H, d, J=3.8 Hz), 2.35 (3H, m), and 1.64 (1H, m) ppm.

Step 9: Potassium 5-(1,1-dioxido-1,2-thiazinan-2-yl)-7-[({4-fluor-
o-2-[(methylamino)carbonyl]benzyl}amino)carbonyl]-1,6-naphthy-
ridin-8-olate

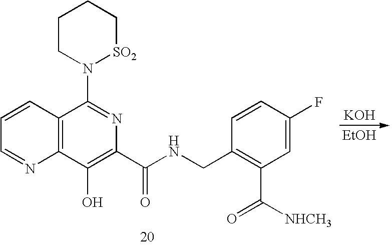

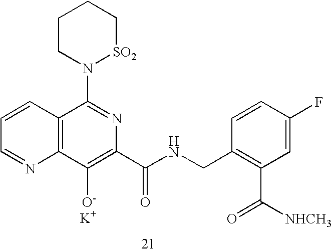

| Material | MW | Equivalents | Amount | Moles |
|---|---|---|---|---|
| carboxamide 20 | 487.1 | 1.0 | 4.2 kg | 8.61 |
| EtOH | | | 20 mL/g 20 | |
| KOH (45 wt. % aq) | 56.1 | 1.2 | 1286 g | 10.34 |
| | | | (866 mL) | |

A 100 L cylinder equipped with a mechanical stirrer, temperature probe, addition funnel, and nitrogen inlet was charged with carboxamide 20 and EtOH (84 L) and then heated to 60° C. To the resulting yellow suspension was added aq KOH. The resulting yellow solution was filtered through a 10 μM line filter into an adjacent 100 L flask. The solution was seeded and heated at 60° C. for 3 hours and then allowed to cool to room temperature overnight. The resulting slurry was cooled to 34° C., filtered, and washed with 4×2 L of cold EtOH. The filter pot was placed under vacuum with a N₂ stream to obtain the title salt as a crystalline ethanolate salt. The purity of the salt was >99.6 A % by HPLC assay. The salt contained 6.8 wt. % ethanol by GC and 0.5 wt. % water by Karl Fisher titration.

### EXAMPLE 5

### Potassium 5-(1,1-dioxido-1,2-thiazinan-2-yl)-7-[({4-fluoro-2-[(methylamino)carbonyl]benzyl}amino)carbonyl]-1,6-naphthyridin-8-olate (Salt A1)

A visually clean 100 L reaction cylinder equipped with an air driven stirrer possessing two propeller blades, a temperature probe, vacuum inlet, and nitrogen inlet was charged with 44 liters of punctillious ethanol (i.e., 200 proof, ethanol with no additives) through a 10 um inline filter. The ethanol solution was placed under nitrogen, heated to 45° C., and then charged with 4.20 kg of free phenol 20 and 40 liters of punctillious ethanol. The resulting slurry was heated to 55° C. and charged with 1.29 kg of 45 wt. % KOH in water. The solution turned homogenous and was subsequently transferred via an 10 um inline filter to an adjacent 100 liter reaction cylinder. The temperature was maintained at 55° C. After about 30 minutes, the solution started to turn hazy as the potassium salt 21 began to crystallize out of solution. The solution was seeded with the 1 g of potassium salt 21 and heating at 58-60° C. was continued. The solution was allowed to cool to room temperature overnight (18 hours). The slurry was then cooled to 4° C., filtered and rinsed with 4×2 liter of punctillious ethanol. The collected solids were dried under vacuum with a purge of nitrogen. A total of 4.62 kg was isolated as the potassium ethanolate salt (95% yield based on free phenol).

The K salt was analyzed by differential scanning calorimetry at a heating rate of 10° C./min from room temperature to 250° C. in an open aluminum pan in a nitrogen atmosphere. The DSC curve exhibited a first endotherm (broad) with a peak temperature of about 69° C. and an associated heat of fusion of about 4 J/gm, a second endotherm (largest, broad) with a peak temperature of about 166° C. and an associated heat of fusion of about 86 J/gm, and a third endotherm with a peak temperature of about 203° C. and an associated heat of fusion of about 4.5 J/gm. Exothermic decomposition was observed above 250° C. No clear melting point was observed.

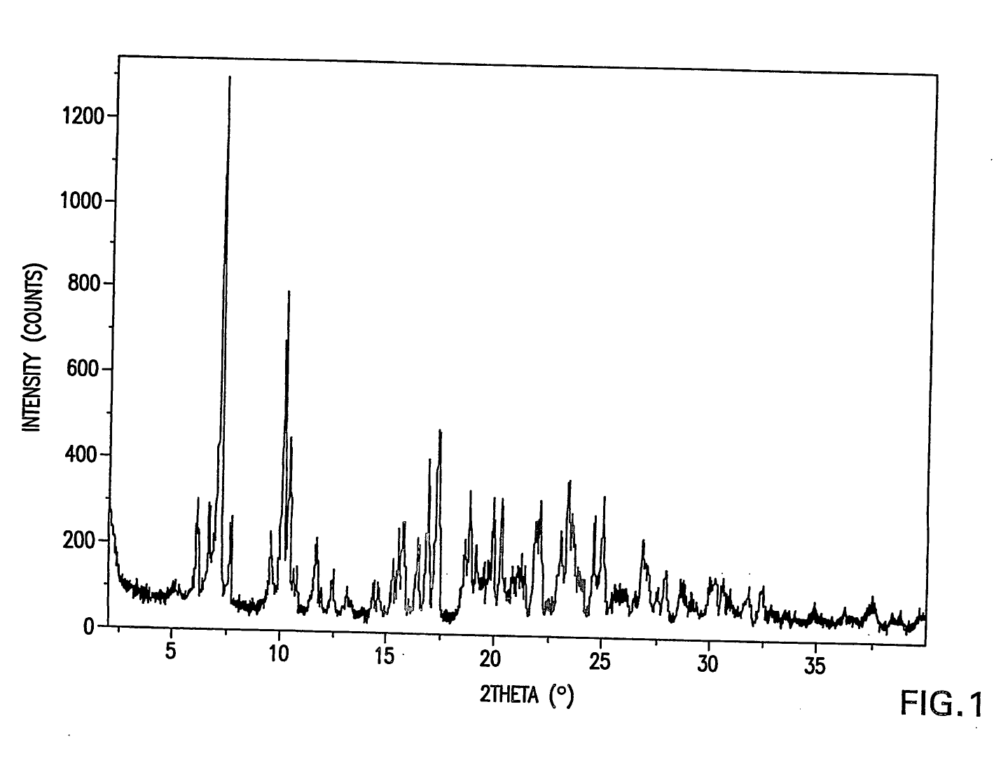

An XRPD pattern of the K salt was generated on a Philips XPert diffractometer using a continuous scan from 2 to 40 degrees 2 theta over about 75 minutes (i.e., 0.0167° step size with 2 seconds/step), 2 RPS stage rotation, and a gonio scan axis. Copper K-Alpha 1 radiation was used as the source. The experiment was run under ambient conditions. The XRPD pattern is shown in FIG. 1. Characteristic d-spacings include the following:

| Peak No. | d-spacing (Å) | 2 Theta |
|---|---|---|
| 1 | 11.88 | 7.432 |
| 2 | 7.45 | 11.867 |
| 3 | 5.07 | 17.474 |
| 4 | 4.68 | 18.939 |
| 5 | 3.29 | 27.084 |
| 6 | 2.96 | 30.150 |

Thermogravimetric analysis (Perkin Elmer Model TGA 7) under a flow of nitrogen at a heating rate of 10° C./minute from room temperature to 250° C. showed that the K salt contain 0.4 wt. % water and 7.2 wt. % ethanol.

### EXAMPLE 6

### Potassium 5-(1,1-dioxido-1,2-thiazinan-2-yl)-7-[({4-fluoro-2-[(methylamino)carbonyl]benzyl}amino)carbonyl]-1,6-naphthyridin-8-olate (Salt A2)

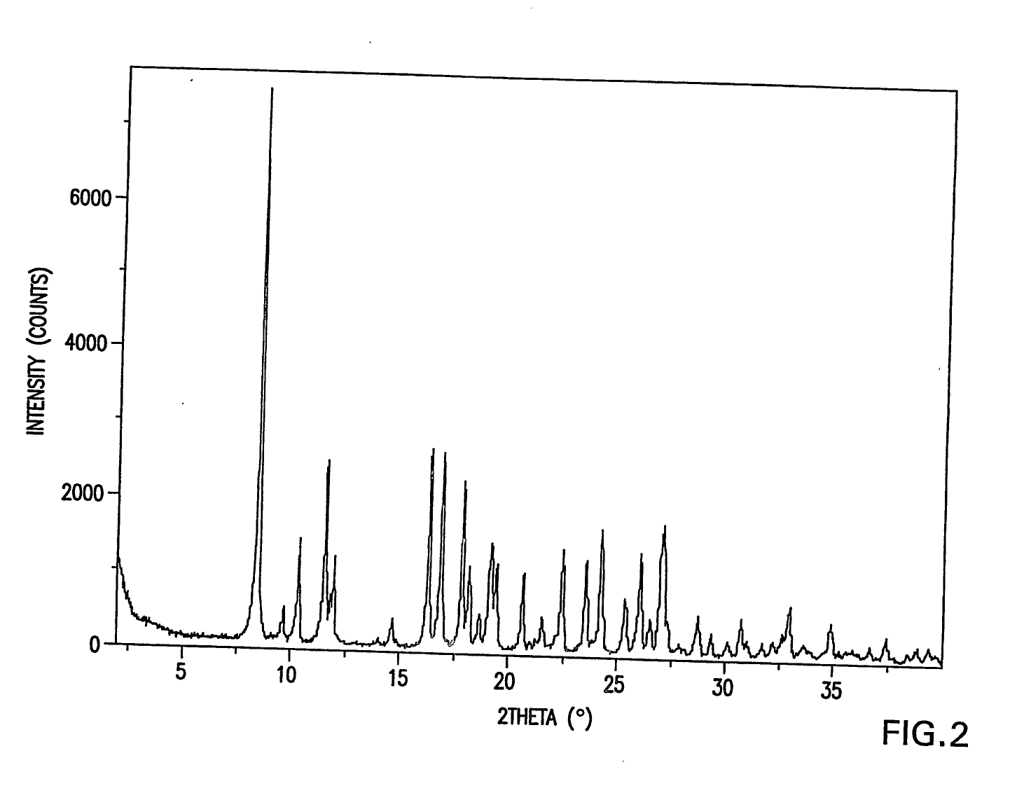

A saturated aqueous solution was prepared using Compound A potassium salt prepared in accordance with Example 5. The saturated solution was allowed to stand undisturbed for 4 months at room temperature, at which point a highly crystalline solid material was isolated from the solution. An XRPD pattern of the isolated potassium salt was obtained using the same instrument and settings employed in Example 5. The XRPD pattern is shown in FIG. 2. Characteristic d-spacings include the following:

| Peak No. | d-spacing (Å) | 2 Theta |
|---|---|---|
| 1 | 10.40 | 8.49 |
| 2 | 10.34 | 8.54 |
| 3 | 5.45 | 16.27 |
| 4 | 5.26 | 16.84 |
| 5 | 3.96 | 22.41 |
| 6 | 3.52 | 25.31 |
| 7 | 2.72 | 32.87 |
| 8 | 2.58 | 34.80 |

The K salt was analyzed by differential scanning calorimetry at a heating rate of 10° C./min from room temperature to 300° C. in a closed pan under a nitrogen atmosphere. The DSC curve exhibited a first endotherm (broad) from about 100 to about 150° C. with a peak temperature of about 144° C. and a second sharp endotherm having an onset temperature of about 270.6° C. and a peak temperature of about 272.4° C. and an associated heat of fusion of about 117 J/gm. The first endotherm is believed to be associated with the desorption of surface adsorbed water remaining from the crystallization, and the second endotherm is believed to be due to melting.

A single crystal X-ray study of the salt was also conducted using a Bruker Smart Apex system at 24° C. and λ=0.71073 angstrom and a 0.08×0.03×0.03 mm crystal. The crystal system was triclinic and the space group P-1. The unit cell dimensions were a=10.195 Å, b=10.892 Å, c=11.426 Å, α=83.822 degrees, β=68.751 degrees, γ=77.16 degrees.

While the foregoing specification teaches the principles of the present invention, with examples provided for the purpose of illustration, the practice of the invention encompasses all of the usual variations, adaptations and/or modifications that come within the scope of the following claims.

1. A potassium salt of Compound A, wherein Compound A is of formula:

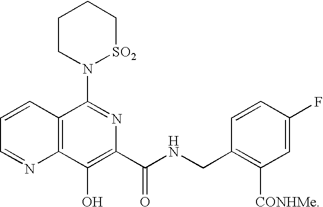

2. The potassium salt according to claim 1, which is a crystalline potassium salt of Compound A.

3. The potassium salt according to claim 2, which is a crystalline potassium salt containing a C₁₋₄ alkyl alcohol as a solvate.

4. The potassium salt according to claim 2, which is a crystalline potassium salt ethanolate of Compound A.

5. The potassium salt according to claim 4, which is a crystalline potassium salt ethanolate hydrate of Compound A.

6. The potassium salt according to claim 5, which is a crystalline potassium salt ethanolate hydrate characterized by containing ethanol in an amount in a range of from about 0.3 to about 7.5 wt. % and water in an amount in a range of from about 0.2 to about 5.5 wt. %

7. The potassium salt according to claim 2, which is an anhydrous, non-solvated crystalline potassium salt.

8. A crystalline monopotassium salt ethanolate of Compound A, characterized by crystallographic d-spacings of 11.88, 7.45 and 5.07 angstroms; wherein Compound A is of formula:

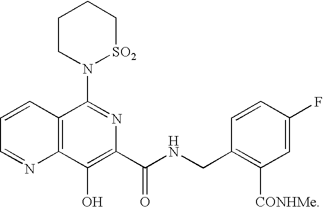

9. The crystalline salt according to claim 8, which is a crystalline monopotassium salt ethanolate hydrate of Compound A, further characterized by a differential scanning calorimetry curve, at a heating rate of 10° C./min in an open cup under nitrogen, exhibiting a first endotherm with a peak temperature of about 69° C. and an associated heat of fusion of about 4 J/gm, a second endotherm with a peak temperature of about 166° C. and an associated heat of fusion of about 86 J/gm, and a third endotherm with a peak temperature of about 203° C. and an associated heat of fusion of about 4.5 J/gm.

10. The crystalline salt according to claim 8, which is a crystalline monopotassium salt ethanolate hydrate of Compound A, further characterized by containing ethanol in an amount in a range of from about 0.3 to about 7.5 wt. % and water in an amount in a range of from about 0.2 to about 5.5 wt. %

11. The crystalline salt according to claim 8, which is characterized by crystallographic d-spacings of 11.88, 7.45, 5.07, 4.68, 3.29 and 2.96 angstroms.

12. The crystalline salt according to claim 11, which is a crystalline monopotassium salt ethanolate hydrate of Compound A, further characterized by a differential scanning calorimetry curve, at a heating rate of 10° C./min in an open cup under nitrogen, exhibiting a first endotherm with a peak temperature of about 69° C. and an associated heat of fusion of about 4 J/gm, a second endotherm with a peak temperature of about 166° C. and an associated heat of fusion of about 86 J/gm, and a third endotherm with a peak temperature of about 203° C. and an associated heat of fusion of about 4.5 J/gm.

13. The crystalline salt according to claim 11, which is a crystalline monopotassium salt ethanolate hydrate of Compound A, further characterized by containing ethanol in an amount in a range of from about 0.3 to about 7.5 wt. % and water in an amount in a range of from about 0.2 to about 5.5 wt. %

14. An anhydrous, non-solvated crystalline monopotassium salt of Compound A, characterized by crystallographic d-spacings of 10.40, 10.34 and 5.45 angstroms; wherein Compound A is of formula:

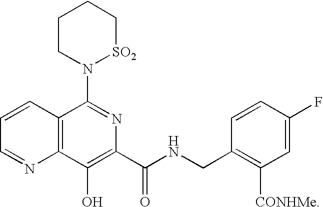

15. The anhydrous, non-solvated crystalline salt according to claim 14, further characterized by a differential scanning calorimetry curve, at a heating rate of 10° C./min in a closed cup under nitrogen, exhibiting a sharp endotherm with an onset temperature of about 270° C., a peak temperature of about 272° C., and an associated heat of fusion of about 117 J/gm.

16. The anhydrous, non-solvated crystalline salt according to claim 14, which is characterized by crystallographic d-spacings of 10.40, 10.34, 5.45, 5.26, 3.96 and 3.52 angstroms.

17. The anhydrous, non-solvated crystalline salt according to claim 16, further characterized by a differential scanning calorimetry curve, at a heating rate of 10° C./min in a closed cup under nitrogen, exhibiting a sharp endotherm with an onset temperature of about 270° C., a peak temperature of about 272° C., and an associated heat of fusion of about 117 J/gm.

18. The anhydrous, non-solvated crystalline salt according to claim 14, which is characterized by crystallographic d-spacings of 10.40, 10.34, 5.45, 5.26, 3.96, 3.52, 2.72 and 2.58 angstroms.

19. The anhydrous, non-solvated crystalline salt according to claim 18, further characterized by a differential scanning calorimetry curve, at a heating rate of 10° C./min in a closed cup under nitrogen, exhibiting a sharp endotherm with an onset temperature of about 270° C., a peak temperature of about 272° C., and an associated heat of fusion of about 117 J/gm.

20. A pharmaceutical composition comprising a therapeutically effective amount of a potassium salt of Compound A as recited in claim 1 and a pharmaceutically acceptable carrier.

21.-28. (canceled)
